(19) United States
(12) Patent Application Publication
(10) Pub. No.: US 2023/0138599 A1
(43) Pub. Date: May 4, 2023

(54) METHOD AND DEVICE FOR ASSISTING AN INVASIVE PROCEDURE ON A HUMAN OR ANIMAL ORGAN

(71) Applicant: PYTHEAS NAVIGATION, MARSEILLE (FR)

(72) Inventor: Yann GLARD, MARSEILLE (FR)

(21) Appl. No.: 17/912,434

(22) PCT Filed: Mar. 18, 2021
(86) PCT No.: PCT/EP2021/056898

§ 371 Date: Sep. 16, 2022

(30) Foreign Application Priority Data
Mar. 18, 2020 (FR) FR2002648

(51) Int. Cl. A61B 34/10; A61B 17/70; G06T 17/00; G06T 19/00

(52) U.S. Cl. CPC A61B 34/10; A61B 17/7001; G06T 17/00; G06T 19/00; A61B 2034/105; A61B 2034/107; A61B 2034/108; G06T 2210/41

(57) ABSTRACT

Disclosed is a method for assisting with an invasive procedure on a human or animal organ, the procedure including inserting at least one member into the organ, the method including obtaining a three-dimensional model of at least part of the organ, determining, on the three-dimensional model obtained, at least one entry point of the at least one member and determining an insertion axis for inserting the at least one member into the organ, according to the three-dimensional model, the at least one determined entry point and a plurality of distinct axes passing through the at least one entry point.

### CROSS-REFERENCE TO RELATED APPLICATION(S)

This application is the U.S. national phase of International Application No. PCT/EP2021/056898 filed Mar. 18, 2021 which designated the U.S. and claims priority to FR 2002648 filed Mar. 18, 2020, the entire contents of each of which are hereby incorporated by reference.

### BACKGROUND OF THE INVENTION

### Field of the Invention

The present invention relates to a method and device for assisting with an invasive procedure on a human or animal organ, for example to plan the putting in place of spinal implants.

The present invention may be used, in particular, for surgery in relation with a substantially rigid human or animal anatomical part (that is to say an anatomical part which does not deform, or deforms only a little, when pressed moderately). By way of illustration, embodiments of the invention concern preparation for spinal surgery, for example preparation for inserting pedicle screws into one or more vertebrae.

### Description of the Related Art

The internal skeletal structure of a mammal, human or animal, is composed of about a hundred bones. The spinal column is a chain of bones called vertebrae allowing a certain flexibility and movements, while protecting the nerve and vascular structures inside and around the spinal column. The vertebral column begins at the base of the skull and extends to the pelvis. It is composed of four regions: cervical, thoracic, lumbar and sacral.

FIGS. 1A and 1B respectively show a top view and a side view of a thoracic vertebra. As illustrated, the vertebra 1 comprises a vertebral body 2 orientated towards the front, a vertebral foramen 3 in the form of a hole allowing the spinal cord to pass through, two transverse processes 4A, 4B, orientated towards the back and outwards, a spinous process 5 between the transverse processes 4A, 4B and orientated downwards, two laminae 6A, 6B which connect the transverse processes 4A, 4B to the spinous process 5, two pedicles 7A and 7B which connect the vertebral body 2 to the transverse processes 4A and 4B, two upper articular facets 8A (not shown) and 8B and two lower articular facets 9A (not shown) and 9B which allow articulation of the vertebra 1 with an adjacent vertebra (they are stacked on each other).

The normal or ideal vertebral alignment can be disturbed due to a trauma or a disease (for example scoliosis). The vertebrae can then pivot about three axes (X, Y, Z), sometimes requiring a surgical procedure in order to correct the anomalies and re-establish an ideal, or at the very least a better, vertebral alignment.

In this case at least two adjacent vertebrae are generally fused to each other by a method in which a surgeon opens the patient, generally from the back, determines an entry point 10 and bores holes 11 in the pedicles 7A and 7B of the vertebra 1 and of one or more other neighboring vertebrae. The holes are bored with an axial angle alpha α (the angle relative to the vertical plane XZ) and a sagittal angle beta β (the angle relative to the plane XY), as illustrated in FIGS. 1A and 1B respectively.

Pedicle screws 12 comprising U-shaped ends 13 (but potentially with other shapes) may then be inserted into the holes 11. For reasons of clarity in FIG. 1A, only a single entry point 10, hole 11, pedicle screw 12, and end 13 are shown.

Each end 13 receives a linking member (not shown), for example a rod, which enables several screws (of several vertebrae) to be connected together and thus to reduce deformation and fuse vertebrae together. The drilled holes 11 and the pedicle screws 12 placed in the vertebrae 1 must be carefully positioned and aligned in order not to injure the adjacent nerve and vascular structures, or even cause the death of the patient. In case of improper positioning of the screws, it is necessary to perform a second operation, giving rise to additional costs and risks.

Guidance systems have been developed to aid the surgeon to bore the holes 11 in the vertebra 1 and accurately place the pedicle screws 12.

Moreover, there are solutions for determining, during a pre-operative phase, the desirable positions for the pedicle screws. Such solutions are generally based on x-ray images, enabling the practitioner to determine an optimum theoretical orientation and position for each screw.

Thus, for example, the paper entitled “A novel cost-effective computer-assisted imaging technology for accurate placement of thoracic pedicle screw”, Y. Abe et al., J Neurosurg: Spine, volume 15, November 2011, discloses a solution for assisting a practitioner in placing pedicle screws. According to this solution, the position and the orientation of a pedicle screw are determined in a step of 3D analysis of data obtained during a pre-operative phase. After having defined this position and this orientation, the entry point for the pedicle screw into the vertebra is deduced therefrom. By using reference points on the vertebra (for example, the transverse processes, the spinous processes and/or the pedicles), the practitioner can, during the operation, determine the entry point on the vertebra and, according to the entry point so determined and the orientation of the pedicle screw determined in the pre-operative phase, bore the vertebra to put in place the pedicle screw.

However, although such methods enable pedicle screws to be put in place in a simplified and economical way, they do not always enable the desired accuracy to be attained.

There is thus a need for a method and device for assisting with an invasive procedure on a substantially rigid human or animal organ, for example to plan the putting in place of spinal implants, making it possible to improve the spatial accuracy of the procedure.

The present invention addresses this issue.

### SUMMARY OF THE INVENTION

To that end, the invention provides for determining an entry point of a member, on a 3D model of the organ in which that member is to be inserted, then for determining the orientation in which that member is to be inserted in order to optimize the position and orientation of that member.

There is thus provided a method for assisting with an invasive procedure on a human or animal organ, said procedure comprising inserting at least one member into said organ, the method comprising (1) obtaining a three-dimensional model of at least part of said organ, (2) determining, on the three-dimensional model obtained, at least one entry point of said at least one member and (3) determining an insertion axis for inserting said at least one member into said organ, according to said three-dimensional model, said at least one determined entry point and a plurality of distinct axes passing through said at least one entry point.

The method according to the invention thus enables accurate positioning of a member by first of all selecting the entry point for that member according to criteria which, in practice, ensure easy determination of that entry point, then by selecting the orientation in which it is to be inserted.

According to a particular embodiment, the method further comprises constructing said three-dimensional model. Such constructing may, for example, be carried out using computed tomography.

Still according to a particular embodiment, the method further comprises displaying a representation of at least part of said three-dimensional model, the displaying at least part of said three-dimensional model enabling the displaying and/or the determining of at least one entry point.

Still according to a particular embodiment, at least two entry points are determined, said plurality of axes being defined by the intersection of a first plane comprising a straight line passing through said at least two entry points and a second plane comprising an entry point. Said second plane may comprise a straight line perpendicular to said first plane.

Still according to a particular embodiment, the method further comprises displaying a cross-section view of said three-dimensional model on said first plane, said first plane forming a first angle relative to a first reference linked to said three-dimensional model.

Still according to a particular embodiment, the method comprises modifying the value of said first angle, the displaying of a cross-section view on said first plane being adapted to said modifying of said first angle.

Still according to a particular embodiment, the method further comprises displaying a cross-section view of said three-dimensional model in said second plane, said second plane forming a second angle relative to a second reference linked to said three-dimensional model.

Still according to a particular embodiment, the method comprises modifying the value of said second angle, displaying a cross-section view in said second plane being adapted to said modifying of said second angle.

Still according to a particular embodiment, the method comprises moving at least one entry point defined on said three-dimensional model.

Still according to a particular embodiment, the method comprises measuring a diameter and/or a length of a member to insert according to a determined entry point, a determined insertion axis and said three-dimensional model.

Said member is, for example, a vertebra and said member to insert is, for example, a pedicle screw.

The invention also relates to a device for assisting with an invasive procedure on a human or animal organ, the device comprising a treatment unit configured to perform each of the steps of the method described above.

A computer program, implementing all or part of the method described above, installed on pre-existing hardware, is itself advantageous, when it provides assistance to help a user identify a desired position for a member in an organ.

Thus, the present invention also relates to a computer program comprising instructions for implementing the method described above, when that program is executed by a processor.

This program may use any programming language (for example an object language or other language) and be in the form of interpretable source code, a partially compiled code or a fully compiled code.

Another aspect relates to a non-transient medium for storing a computer-executable program, comprising a set of data representing one or more programs, said one or more programs comprising instructions for carrying out all of or part of the method described above on executing said one or more programs by a computer comprising a processing unit coupled operatively to memory means and to an input/output interface module.

### BRIEF DESCRIPTION OF THE DRAWINGS

Other features, details and advantages of the invention will appear on reading the following detailed description. This description is purely illustrative and must be read with regard to the accompanying drawings, in which:

FIG. 1A and FIG. 1B respectively show a top view and a side view of a thoracic vertebra.

FIG. 2 illustrates an example of steps which can be implemented in a tool for assisting with an invasive procedure on a human or animal organ according to the invention;

FIG. 3 illustrates an example of steps for obtaining a 3D model of a human or animal organ or of a portion thereof;

FIG. 4 illustrates an example of steps for determining an axis for inserting a member into a human or animal organ, for example an axis of a mounting such as a pedicle screw;

FIG. 5A and FIG. 5B illustrate an example of a 3D model of a vertebral column, enabling a user to determine one or more entry points then to determine one or more insertion axes for inserting pedicle screws;

FIG. 6A and FIG. 6B show an example of a graphical interface which can be used by a user to determine entry points and insertion axes for inserting members such as pedicle screws;

FIG. 7 diagrammatically illustrates an example of a solution for determining an insertion axis for inserting a member into an organ, for example a pedicle screw into a vertebra;

FIG. 8 illustrates an example of cross-section views obtained on rotating a plane passing through two entry points, according to the mechanism illustrated in FIG. 7;

FIG. 9 illustrates an example of cross-section views obtained on rotating a plane perpendicular to a plane passing through two entry points, according to the FIG. 10

FIG. 10 shows an example of a device for processing data making it possible to implement the method according to embodiments of the invention.

### DESCRIPTION OF THE PREFERRED EMBODIMENTS

According to embodiments, a tool for assisting with an invasive procedure on a human or animal organ according to the invention is first of all directed to determining an entry point into the organ, then, from that entry point, to determining a position and an advantageous orientation for a member to insert. By selecting an entry point that can be easily identified at the time of the procedure, it is possible to accurately position a member in the organ. As a matter of fact, the inventors have noted that it was often preferable to select a non-optimum entry point and to accurately position a member rather than determine an optimum position for a member and to deduce therefrom an entry point which, in practice, may prove difficult to determine.

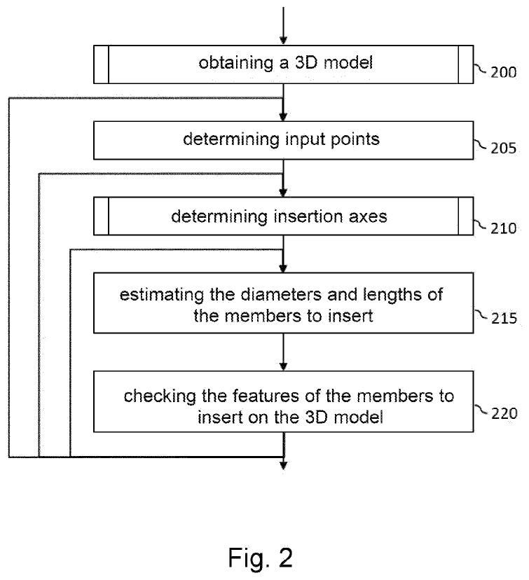

FIG. 2 illustrates an example of steps which can be implemented in a tool for assisting with an invasive procedure on a human or animal organ according to the invention.

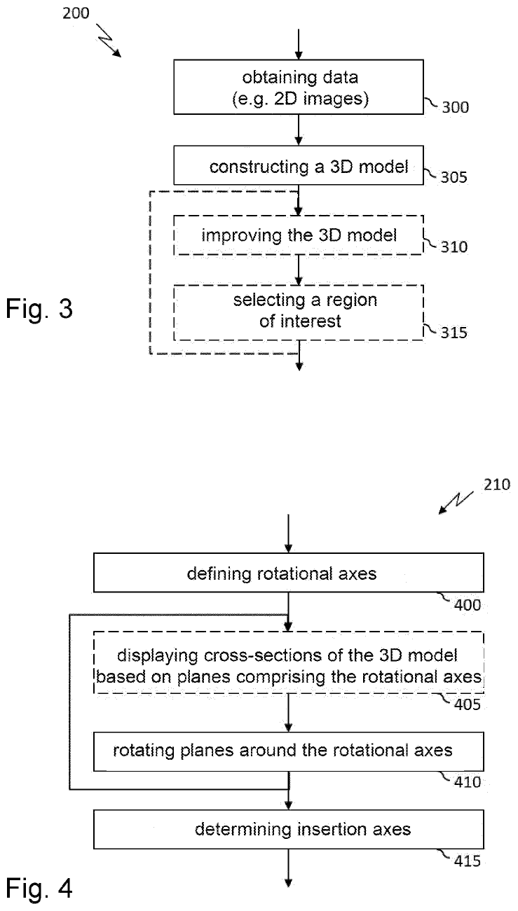

As illustrated, a first step is directed to obtaining a three-dimensional digital model (called 3D model below) of a human or animal organ, or of a portion thereof, in which the procedure is to take place (step 200). An example of obtaining such a 3D model will be described with reference to FIG. 3. According to embodiments, this is a model enabling surface viewing, that is to say of an envelope of the organ considered, comprising its outside face and, as the case may be, its orifices or recesses. Naturally, the 3D model may be more complex and comprise other information such as representations of the nerve endings, blood vessels, etc.

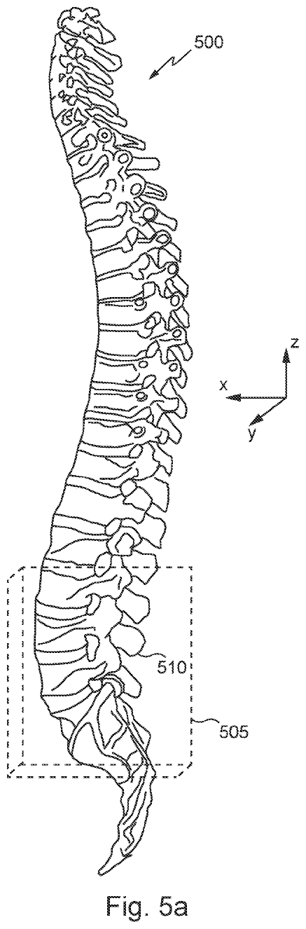

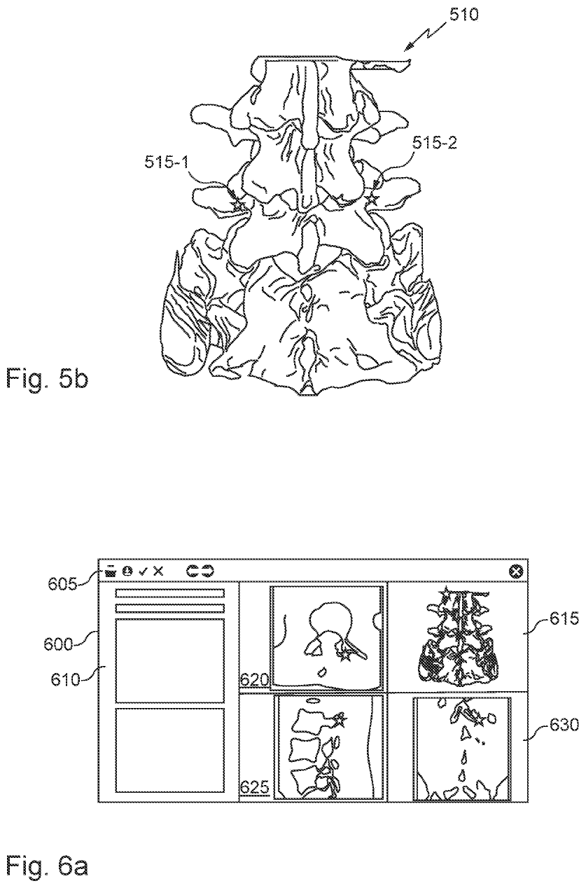

In a following step, one or more entry points are determined on the 3D model (step 205) or based thereon, for example on cross-section views of the 3D models. They may be determined automatically or by a user, for example on a graphical representation of the 3D model or of a portion of that model. When the entry point or points have been determined by a user on a graphical representation of the 3D model, a viewing application that enables the viewing angle of the organ to be changed is preferably used. Such an application is able to perform operations of change of scale, rotation about several axes and movement in several directions. An example of representations of a 3D model and of a portion of that model is illustrated in FIGS. 5A and 5B. These representations make it possible to determine an entry point. As illustrated, it is, preferably, possible to select a portion of the model (also called region of interest), for example using a cube, a cylinder or any other three-dimensional shape of which, in particular, the shape, the size, the position and/or the orientation may be adjusted by a user or automatically.

According to other embodiments, an entry point is determined from cross-section views of the 3D model, for example from two cross-section views on perpendicular planes comprising a same point that can be selected as an entry point. According to these embodiments, this point which can be selected as an entry point may be moved over the surface of the 3D model, that is to say on cross-section lines of the cross-section views, for example using a mouse wheel or keyboard keys. According to other embodiments, the entry point or points may be selected on the surface of the 3D model and/or in the organ, under the surface of the 3D model and in the vicinity of the latter. As a matter of fact, it can occur, in practice, that a practitioner will eliminate part of the organ considered, for example a piece of bone, to select one or more entry points.

To automatically determine an entry point, an analysis of the 3D model or of a portion of the 3D model may be made to identify notable points, that are easy to identify during a procedure, and from which the entry point may be defined. Such notable points are, for example, the transverse processes and the spinous process. By way of illustration, such an analysis may be made by an artificial intelligence algorithm trained with 3D models on which notable points have been placed by a user or have been validated by a user.

In a following step, the position and the orientation of one or more members to be inserted into the organ are determined, based on the entry point or points already selected (step 210). According to embodiments, such position and orientation correspond to an axis of a mounting, extending from the entry point. This position and this orientation may be determined automatically from predetermined constraints or be determined by a user, for example on a graphical representation of the 3D model or of the portion of that model. An example of determining a position by a user is described with reference to FIGS. 7 to 9.

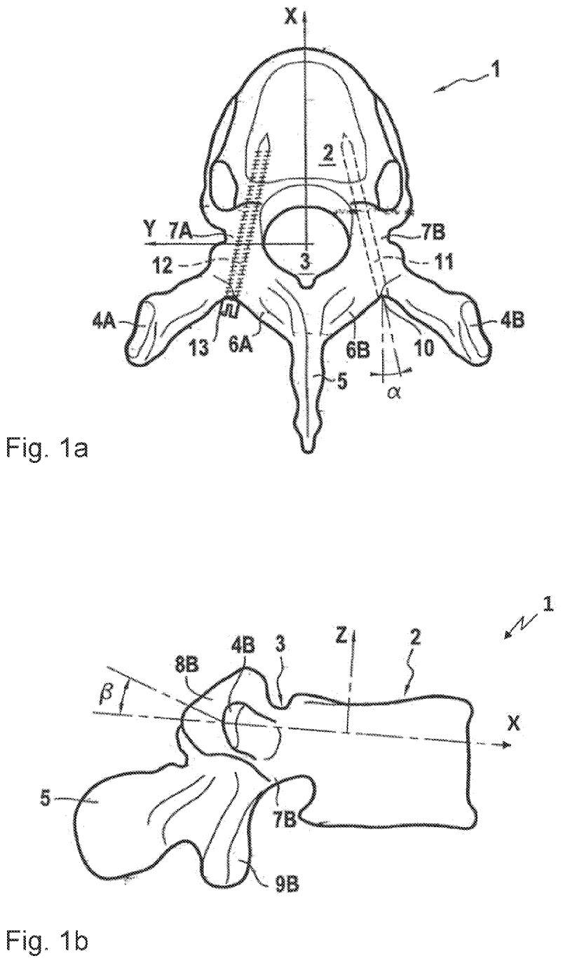

After the position and the orientation of the member or members to be inserted has been determined, the features of those members are, as the case may be, estimated (step 215). This may, for example, comprise determining the diameter and the length of the members to use, which latter may, by way of illustration, be pedicle screws. These features may be determined automatically or may be determined by a user. When they are determined by a user, a check is, preferably, made (step 220). Such a check may in particular comprise displaying a representation of the members on a representation of the organ, at the same scale, to enable the user to check the suitability of the members in relation to the organ. These representations may be similar to those illustrated in FIG. 1A. Again, an application enabling the display and the manipulation of digital objects makes it possible, preferably, to modify the display (viewing angle, scale, section plane, etc.).

As illustrated, it is possible to modify the position of the entry point or points (return to reference 205), the position and orientation of the members to be inserted (return to reference 210), for example to refine those positions and orientations, or to re-estimate the features of those members (return to reference 215).

The steps illustrated in FIG. 2 thus make it possible, based on an entry point, to determine a position and an orientation that meet predetermined constraints. As the entry point can be selected so as to be easily identifiable at the time of the procedure, the tool for assisting with an invasive procedure according to the invention provides advantages in terms of accuracy.

FIG. 3 illustrates an example of steps for obtaining a 3D model of a human or animal organ or of a portion thereof.

According to the illustrated example, a first step is directed to obtaining a set of data, for example of images, in cross-section, of the human or animal organ, or of part thereof, for which the 3D model is sought (step 300). These data may be obtained using a scanner, for example an X-ray scanner, using a digital tomography technology, known as computed tomography (CT).

These data are next combined to obtain a 3D model according to predetermined criteria, for example to obtain a 3D model representing the outside surface be the organ. Numerous solutions exist for constructing a 3D model from computed tomography data (or CT data). The papers known under the following references Ney D R, Fishman E K, Magid D, Robertson D D, Kawashima A “Three-dimensional volumetric display of CT data: effect of scan parameters upon image quality”, JComput Assist Tomogr, 1991 September-October; 15(5):875-85, PubMed PMID: 1885819, Rothman S L, Geehr R B, Kier E L, Hoffman H B “Multiplanar reconstruction as an aid in CT diagnosis”, Neuroradiology, 1978; 16:596-7, PubMed PMID: 745768 et Aubry S, Pousse A, Sarliève P, Laborie L, Delabrousse E, Kastler B, “Three-dimensional 3D modeling: First applications in radioanatomy and interventional radiology under CT guidance”, J Radiol, 2006 November; 87 (11 Pt 1):1683-9, French PubMed PMID: 17095963 describe some of these methods or some elements of these methods.

According to embodiments, the 3D model obtained may be improved (step 310), for example to eliminate noise. Examples of improving a 3D model are presented in the paper referenced Bibb, R. (2006) “Medical imaging for rapid prototyping”, MedicalModelling, 8-31, doi:10.1533/9781845692001.8

As described above, a region of interest of a 3D model may, optionally, be selected (step 315), automatically according to predetermined criteria or manually by a user. By way of illustration, if the 3D model corresponds to a vertebral column, a region of interest my correspond to one or more vertebrae, as illustrated in FIGS. 5A and 5B.

According to embodiments, the region of interest is checked (step not shown in FIG. 3) to verify that the aforementioned comprises discriminating elements making it possible to identify a point of interest. By way of illustration, it may thus be checked that a portion of a 3D model of a vertebral column comprises at least the second cervical vertebra (C2), the first or the twelfth thoracic vertebra (T1 or T12) or the fifth lumbar vertebra (L5).

FIG. 4 illustrates an example of steps for determining an axis for inserting a member into a human or animal organ, for example an axis of a mounting such as a pedicle screw;

A first step is directed here to defining a variable directly or indirectly controlling the direction of the angle of insertion of the member into the organ (step 400). By way of illustration, this direction may be defined by the intersection of two planes, for example a first plane comprising a straight line linking two entry points and a second plane comprising a straight line perpendicular to the first plane and comprising one of these two entry points. In this case, the variable may be defined by two angles associated with two rotational axes, one of these two rotational axes being the straight line passing through the two entry points and the other rotational axis being the straight line perpendicular to the first plane and passing through one of the entry points. These angles are defined in relation to a predetermined reference. These two rotational axes are illustrated in FIG. 7.

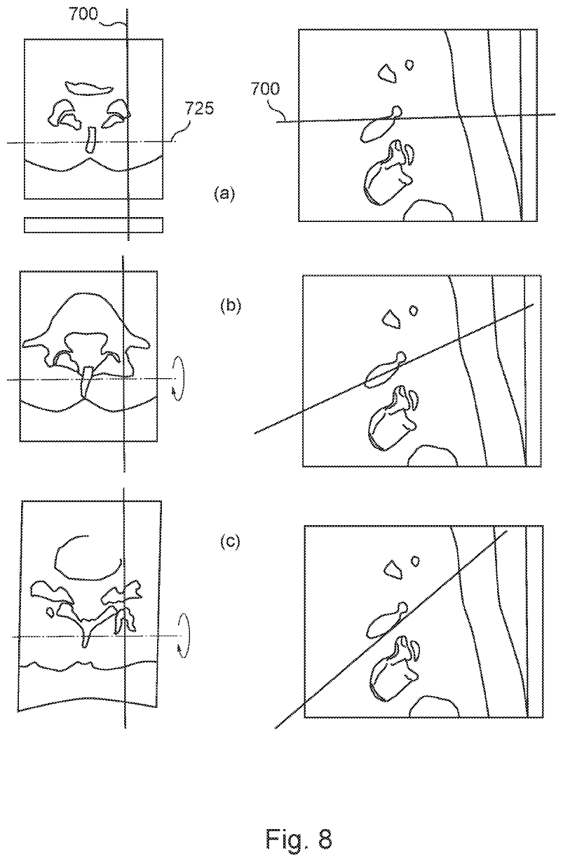

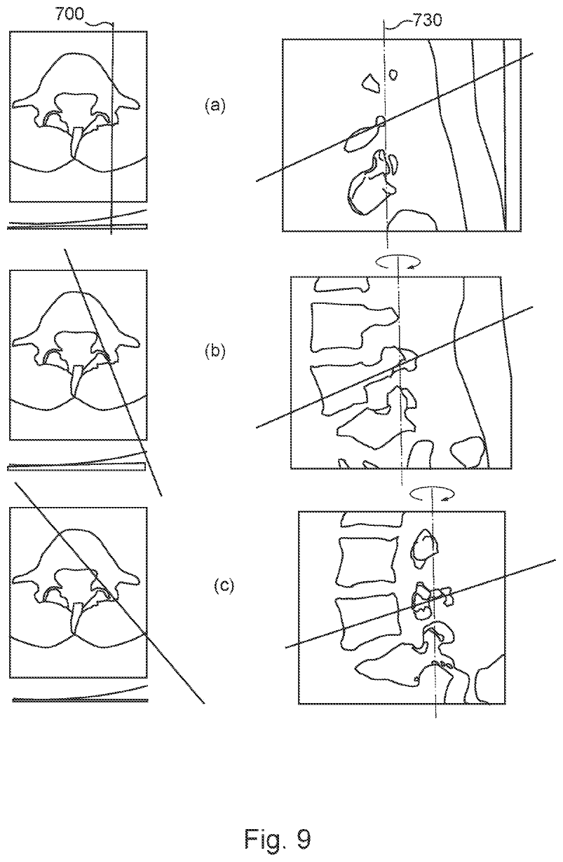

In a following step, one or more cross-section views of the 3D model may be displayed to assist a user to choose a direction for the insertion axis of the member into the organ (step 405). They may, for example, be cross-section views on the first and second planes defined above. An example for such representations is illustrated in FIGS. 8 and 9. The user may thus vary the section planes (step 410), for example by turning them around the rotational axes described above, for example using a mouse wheel (it being possible, for example, for one of the rotations to be made directly with the mouse wheel and the other by simultaneously pressing a key, for example the shift key). When the user is satisfied with the direction of the insertion axis for the member into the organ, he or she can validate it (step 415).

The process for determining the axes for inserting members into the organ may also be automatic, for example by varying the variable or variables directly or indirectly controlling the axis for inserting a member into an organ between given values and by identifying an optimum position according to predetermined criteria or a position meeting predetermined criteria.

By way of illustration, the rotational angle of the first plane comprising the straight line passing through two entry points can vary from −θ to θ, for example from −35° to 35°, relative to a reference plane (for example a horizontal plane in the case of pedicle screws). Similarly, the rotational angle of the second plane, comprising a straight line perpendicular to the first plane, can vary from −φ to φ, for example from −20° to 20°, relative to a reference plane (for example a vertical plane perpendicular the first plane).

FIGS. 5A and 5B illustrate an example of a 3D model of a vertebral column, enabling a user to determine one or more entry points then to determine one or more insertion axes for inserting pedicle screws.

As illustrated, the model is associated here with a frame of reference, for example a Cartesian coordinate system in which the x-axis is a horizontal axis extending forward, the y-axis is a horizontal axis perpendicular to the x-axis and the z-axis is a vertical axis extending upward. Naturally, another frame of reference may be used, for example a frame of reference linked to a vertebra. According to one embodiment, the 3D model is represented on a screen, as illustrated in FIG. 5A. Preferably, there are functions making it possible to move the 3D model on the screen, to modify its orientation and/or to modify its scale.

According to a particular embodiment, a user may select a region of interest, for example using a parallelepiped 505, a cylinder or any other three-dimensional shape, of which, in particular, the shape, the size, the position and/or the orientation can be adjusted by a user or automatically. The portion of the 3D model 510 so defined may be displayed, as illustrated in FIG. 5B. Again, the user may, preferably, move that portion on the screen, modify its orientation and/or modify its scale. He or she may also, preferably, execute an image processing algorithm to improve the rendering quality of the representation, for example to reduce noise.

The representation of the 3D model or of a portion thereof makes it possible to determine one or more entry points, for example the entry points 515-1 and 515-2. Based on these entry points, it is possible to determine the axes for inserting pedicle screws as described in more detail with reference to FIGS. 7, 8 and 9.

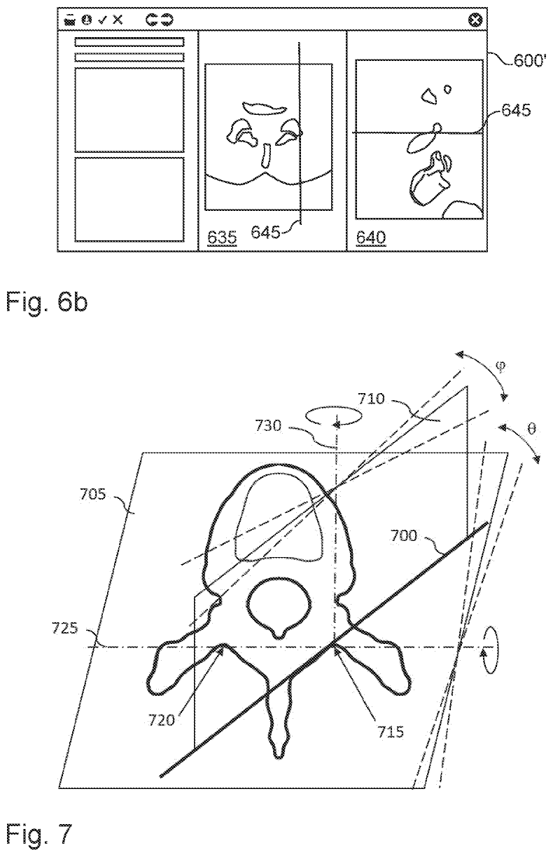

To facilitate determining entry points and/or axes for inserting pedicle screws, several views may be presented simultaneously, as illustrated in FIGS. 6A and 6B.

FIGS. 6A and 6B show an example of a graphical interface which can be used by a user to determine entry points and axes for inserting pedicle screws.

More specifically, FIG. 6A illustrates an example of a graphical interface 600 for determining an entry point. As illustrated, the graphical interface 600 here comprises several zones among which is a zone 605 comprising icons enabling access to menus, a text zone 610 to display or enter, for example, information or instructions and four graphical zones 615 to 630. According to this example, the zone 615 comprises a representation of the 3D model or of a portion thereof while the zones 620, 625 and 630 respectively comprise cross-section views of the 3D model, for example on planes xy, xz and yz comprising a given point which can be moved by a user, for example a point that can be selected by a user as entry point. To move that point, the user can, for example, select one of the zones 620, 625 or 630 and move the point using a mouse, a mouse wheel or keyboard keys (for example the left, right, up and down keys). When the position of the point is modified, the representations on the screen are modified too, advantageously immediately (i.e. in real-time). As described above, this point (here in the form of a star to be visible) may be selected by the user as entry point.

FIG. 6B illustrates an example of a graphical interface 600′ for determining an insertion axis for inserting a member into an organ, for example a pedicle screw into a vertebra. In addition to zones comprising icons or locations for displaying or inputting information or instructions, similar to those shown in FIG. 6A, the graphical interface here comprises two graphical zones 635 and 640.

According to a particular embodiment, each of the zones 635 and 640 comprises a cross-section view of the 3D model, these views being obtained on two planes at a right angle the intersection of which represents the axis for inserting a pedicle screw. Thus, for example, the zone 635 comprises a cross-section view on a first plane comprising two entry points, for example entry points located on two opposite laminae of a same vertebra, while the zone 640 comprises a cross-section view on a second plane, comprising a straight line perpendicular to the first plane and comprising the entry point associated with the insertion axis in course of determination. According to one embodiment, the user can select the zone 635 or the zone 640 and use a wheel or two arrows to increase or decrease the angle of the plane corresponding to the cross-section viewed.

The insertion axis 645 and/or the member to insert are shown in cross-section views to assist the user in determining that axis.

FIG. 7 diagrammatically illustrates an example of a solution for determining an insertion axis for inserting a member into an organ, for example a pedicle screw into a vertebra.

According to this example, an insertion axis is defined by a straight line formed by the intersection of two planes and that comprises an entry point defined earlier for the member to be inserted. As illustrated in FIG. 7, the insertion axis in course of determination is the axis 700 here, resulting from the intersection of the planes 705 and 710, comprising the entry point 715. For reasons of clarity, only plane 705 comprises a representation of a cross-section view of the organ, here a vertebra.

By way of illustration, the plane 705 is a plane comprising the straight line 725 passing through the entry point 715 and another entry point, here the entry point 720. The plane 705 may thus turn around the axis 725 and form an angle θ relative to a reference plane, for example the plane perpendicular to the vertebral column and comprising entry points 715 and 720.

Still by way of illustration, the plane 710 is a plane comprising a straight line 730 perpendicular to the plane 705 and comprising the entry point 715. The plane 720 can thus turn around the axis 730 (perpendicular to the plane 705 and comprising the entry point 715). It can form an angle φ relative to a reference plane, for example the vertical plane perpendicular to the axis 725.

To determine the insertion axis for a member, a user (or an algorithm) can modify the values of the angles θ and φ in order to view (or analyze) the views in cross-section on the two planes defining the insertion axis. As described above, the value of the angles θ and φ can in particular be modified using wheels, keys or a combination of a wheel and a key.

The insertion axis can thus be defined, for example, by the angles θ and φ and reference planes.

FIG. 8 illustrates an example of cross-section views obtained on rotating a plane passing through two entry points, according to the mechanism illustrated in FIG. 7.

The views located on the left part are cross-section views on the plane 705 passing through the entry points 715 and 720 while the views located on the right part are cross-section views on the plane 710, comprising the straight line 730 (perpendicular to the plane 705 and passing through the entry point 715). The groups of views referenced (a), (b) and (c) correspond to different values of the angle θ.

FIG. 9 illustrates an example of cross-section views obtained on rotating a plane perpendicular to a plane passing through two entry points, according to the mechanism illustrated in FIG. 7.

Again, the views located on the left part are cross-section views on the plane 705 passing through the entry points 715 and 720 while the views located on the right part are cross-section views on the plane 710, comprising the straight line 730 (perpendicular to the plane 705 and passing through the entry point 715). The groups of views referenced (a), (b) and (c) correspond to different values of the angle cp.

Thus, by successively varying the angles θ and φ, it is possible to find an insertion axis enabling the insertion of a member into an organ based on an entry point, complying with predetermined criteria. To assist the user, a representation of the member to be inserted may be added to the views in cross-section, the position of this representation being determined by the entry point and the insertion axis considered.

As described above, the values of the angles θ and φ may be modified by a user to enable him or her, based on the cross-section views, to determine the insertion axis which seems optimum to him or her. Alternatively, the values of the angles s and φ may be modified automatically, according to predetermined increments. For each increment, an analysis of the cross-section views is carried out to determine whether the insertion axis defined by the angles s and φ meets predetermined criteria. If these criteria are met, the algorithm terminates or stores in memory the identified values for the purpose, later, of comparing the different insertion axes meeting the predetermined criteria and of selecting the insertion axis having optimum criteria.

According to a particular embodiment, assistance is given to the user to enable him or her to modify the angles θ and φ while indicating whether the current values of these angles meet predetermined criteria. Thus, for example, the insertion axis may be colored green if the insertion axis meets the predetermined criteria and red if those criteria are not met. Similarly, these colors may have higher or lower intensity according to, for example, a quantification of risk associated with a position of the insertion axes.

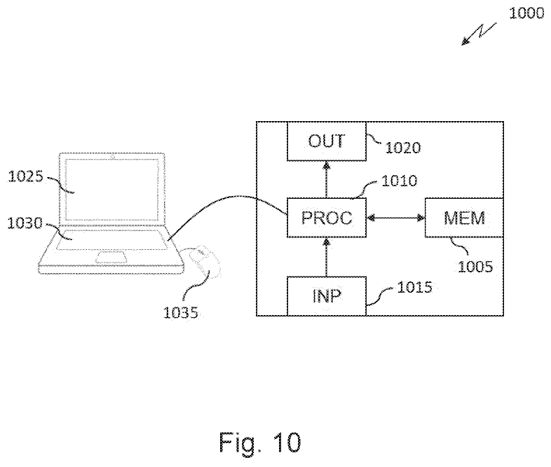

FIG. 10 represents an example of a data processing device enabling implementation of the method according to embodiments of the invention, in particular to execute all or some of the steps described with reference to FIGS. 2, 3 and 4.

According to the illustrated example, the device 1000 comprises a memory 1005 for storing instructions enabling the implementation of the method, the measurement data received and temporary data for performing various steps of the method as described above.

The device further comprises a circuit 1010. The circuit may, for example, be:
a processor able to interpret instructions in computer program form, or
an electronic card in the hardware elements of which steps of the method of the invention are implemented, or for instance
a programmable electronic chip such as an FPGA (for “Field-Programmable Gate Array”), a SOC (for “System On Chip”) or an ASIC (for “Application Specific Integrated Circuit”). SOCs and systems on a chip are embedded systems which integrate all the components of an electronic system in a single chip.

An ASIC is a specialized electronic circuit which groups together functionalities tailored to a given application. ASICs are generally configured at the time of their manufacture.

Programmable logic circuits of FPGA type are electronic circuits that are reconfigurable by a user.

Device 1000 here comprises an input interface 1015 for receiving measurement data, for example CT data enabling the construction of a 3D model, and an output interface 1020. Lastly, to enable easy interaction with a user, it may comprise a screen 1025, a keyboard 1030 and a mouse 1035, preferably provided with a wheel. Of course, the keyboard is optional, in particular in the context of a computer having the form for a touch-screen tablet, for example.

According to the embodiment, the device 1000 may be a computer, a network of computers, an electronic component or another apparatus comprising a processor operatively coupled to a memory, and, according to the embodiment chosen, a data storage unit and other associated hardware components such as a network interface and a medium reader for reading a removable storage medium and to write on such a medium (not shown in the drawing). The removable storage medium may for example be a compact disc (CD), a video disc/digital versatile disc (DVD), a flash disc, a USB memory stick, etc.

According to the embodiment, the memory, the data storage unit or the removable storage medium contains instructions which, when executed by the circuit 1010, lead the circuit 1010 to perform or control parts which are the input interface 1015, output interface 1020, data storage in the memory 1005 and/or data processing.

The functional diagrams presented in FIGS. 2, 3 and 4 are examples of programs of which all or some instructions may be carried out by the device described.

Of course, the present invention is not limited to the embodiments described above by way of example, and extends to other variants. Other embodiments are possible.

According to the embodiment chosen, certain acts, actions, events or functions of each of the methods described in the present document may be carried out or occur in a different order than that in which they have been described, or may be added, merged or not carried out or not occur, according to the case. Furthermore, in some embodiments, certain acts, actions or events are carried out or occur concurrently and not successively.

Although described through a certain number of detailed examples, the method provided and the equipment for the implementation of the method comprise different variants, modifications and improvements which will be obviously apparent to the person skilled in the art, it being understood that these different variants, modifications and improvements form part of the scope of the invention, as defined by the following claims. Furthermore, different aspects and features described above may be implemented together, or separately, or else be substituted for each other, and all the different combinations and sub-combinations of the aspects and features form part of the scope of the invention. Furthermore, it may be that some systems and equipment described above do not incorporate all the modules and functions described for the preferred embodiments.

1. Method for assisting with an invasive procedure on a human or animal organ, said procedure comprising inserting at least one member into said organ, the method comprising:
a. obtaining a three-dimensional model of at least part of said organ;
b. determining, on the three-dimensional model obtained, at least one entry point of said at least one member;
c. determining an insertion axis for inserting said at least one member into said organ, according to said three-dimensional model, said at least one determined entry point and a plurality of distinct axes passing through said at least one entry point, said plurality of axes being defined by the intersection of a first and a second plane, said first and second planes comprising said at least one entry point,
d. displaying a cross-section view of said three-dimensional model on said first plane, said first plane forming a first angle relative to a first reference linked to said three-dimensional model,
e. modifying the value of said first angle, the displaying of a cross-section view on said first plane being adapted to said modifying of said first angle.

2. The method according to claim 1 further comprising constructing said three-dimensional model.

3. The method according to claim 1, comprising displaying a representation of at least part of said three-dimensional model, the displaying at least part of said three-dimensional model enabling the displaying and/or the determining of at least one entry point.

4. The method according to claim 1, wherein at least two entry points are determined, said first plane comprising a straight line passing through said at least two entry points.

5. The method according to claim 4, wherein said second plane comprises a straight line perpendicular to said first plane.

6. The method according to claim 1, further comprising displaying a cross-section view of said three-dimensional model on said second plane, said second plane forming a second angle relative to a second reference linked to said three-dimensional model.

7. The method according to claim 6, wherein the value of said second angle is modified, the displaying of a cross-section view on said second plane being adapted to said modifying of said second angle.

8. The method according to claim 1, comprising moving at least one entry point defined on said three-dimensional model.

9. The method according to claim 1, comprising measuring a diameter and/or a length of a member to insert according to a determined entry point, a determined insertion axis and said three-dimensional model.

10. The method according to claim 1, wherein said member is a vertebra and said member to insert is a pedicle screw.

11. (canceled)

12. Computer-readable non-transient recording medium on which is recorded a program for implementing each of the steps of the method according to claim 1, when that program is executed by a processor.

13. Device comprising a computing unit configured for executing each of the steps of the method according to claim 1.

14. The method according to claim 2, comprising displaying a representation of at least part of said three-dimensional model, the displaying at least part of said three-dimensional model enabling the displaying and/or the determining of at least one entry point.

15. The method according to claim 2, wherein at least two entry points are determined, said first plane comprising a straight line passing through said at least two entry points.

16. The method according to claim 3, wherein at least two entry points are determined, said first plane comprising a straight line passing through said at least two entry points.

17. The method according to claim 2, further comprising displaying a cross-section view of said three-dimensional model on said second plane, said second plane forming a second angle relative to a second reference linked to said three-dimensional model.

18. The method according to claim 3, further comprising displaying a cross-section view of said three-dimensional model on said second plane, said second plane forming a second angle relative to a second reference linked to said three-dimensional model.

19. The method according to claim 4, further comprising displaying a cross-section view of said three-dimensional model on said second plane, said second plane forming a second angle relative to a second reference linked to said three-dimensional model.

20. The method according to claim 5, further comprising displaying a cross-section view of said three-dimensional model on said second plane, said second plane forming a second angle relative to a second reference linked to said three-dimensional model.

21. The method according to claim 2, comprising moving at least one entry point defined on said three-dimensional model.
